(19) United States
(12) Patent Application Publication
(10) Pub. No.: US 2008/0242992 A1
(43) Pub. Date: Oct. 2, 2008

(54) Ultrasound Imaging System and Method for Flow Imaging Using Real-Time Spatial Compounding

(75) Inventor: Aline Criton, Seattle, WA (US)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 12/065,153

(22) PCT Filed: Aug. 30, 2006
(86) PCT No.: PCT/IB06/53023

§ 371 Date: Feb. 28, 2008

Related U.S. Application Data
(60) Provisional application No. 60/713,182, filed on Aug. 31, 2005.

(51) Int. Cl. A61B 8/00

(52) U.S. Cl. 600/447

(57) ABSTRACT

A method for reducing speckle in an ultrasound image includes generating a transmit scan beam from a single aperture defined on a face of a transducer element array, such that the transmit scan beam originates from the single aperture, generating a first set of ultrasound response scan beams, originating from a first receive aperture, defined as a first set of transducer elements symmetrically across the center of the transmit aperture, generating at least a second set of ultrasound response scan beams, originating from at least a second receive aperture contiguous with the first receive aperture. The at least second receive aperture is defined by at least a second set of transducer elements disposed symmetrically across the center of the transmit aperture. The response scan beams are received simultaneously by the first and the at least second receive apertures, and compounded.

The present invention is generally related to ultrasound imaging systems, and more particularly, to an ultrasound imaging system, and an imaging method, which employ real-time spatial compounding in flow imaging, i.e., color flow and CPA, to reduce speckle without compromising frame rate.

Ultrasonic imaging has become an important and popular diagnostic tool with a wide range of applications. Particularly, due to its non-invasive, and typically non-destructive nature, ultrasound imaging has been used extensively in the medical profession. Modern high-performance ultrasound imaging systems and techniques are commonly used to produce two-dimensional (2D) and three dimensional (3D) diagnostic images of internal features of an object (e.g., portions of the anatomy of a human patient). A diagnostic ultrasound imaging system generally uses a wide bandwidth transducer to emit and receive ultrasound signals. That is, the imaging system forms images of the internal tissues of a human body by electrically exciting an acoustic transducer element, or an array of acoustic transducer elements, to generate ultrasonic pulses that travel into the body. The ultrasonic pulses generate echoes as they reflect off of body tissues that appear as discontinuities to the propagating ultrasonic pulses. The various echoes return to the transducer and are converted into electrical signals that are amplified and processed to produce an image of the tissues.

The ultrasonic (acoustic) transducer, which radiates the ultrasonic pulses, typically comprises a piezoelectric element or an array of piezoelectric elements. As is known in the art, a piezoelectric element deforms upon application of an electrical signal to produce the transmitted ultrasonic pulses. Similarly, the received echoes cause the piezoelectric element to deform and generate a corresponding receive electrical signal. The acoustic transducer is often packaged in a handheld device that allows an operator substantial freedom to manipulate the transducer over a desired area of interest. The transducer is often connected via a cable to a control device that generates and processes the electrical signals. In turn, the control device may transmit image information to a real-time viewing device, such as a display monitor. In alternative configurations, the image information may also be transmitted to physicians at a remote location and or stored in a recording device to permit viewing of the diagnostic images at a later time.

One fundamental problem in all types of ultrasound imaging is noise from back-scattered signals, which obscures the details of the target image or echo. One type of noise, commonly known as “speckle,” results from constructive and destructive interference, and appears as a random mottle superimposed on the image. Normally, speckle is received from objects having dimensions smaller than the wavelengths generated by the ultrasound energy source, making it impossible to reduce the speckle simply by increasing the resolution of the device. Moreover, speckle originates from objects that are stationary and randomly distributed. Since the speckle has no phase or amplitude variation over time, one cannot suppress the speckle by averaging the image signals over time. In other words, speckle signals are coherent and cannot be reduced by time averaging.

One way to reduce speckle noise is through a method known as spatial compounding. Spatial Compounding reduces noise, improves the visualization of specular interfaces, and reduces shadowing artefacts. Spatial compounding imaging combines a number of ultrasound images of a given target that have been obtained from a multiple vantage points or angles into a single compounded image (U.S. Pat. Nos. 4,649,927; 4,319,486; 4,159,462; etc.). In B-mode imaging, spatial compounding has proved to be an effective technique for reducing speckle noise, improving the visualization of specular interfaces, and reducing shadowing artefacts (Trahey, Smith et al. 1986; Trahey, Smith et al. 1986; Silverstein and O'Donnell 1987; O'Donnell and Silverstein 1988).

Doppler imaging techniques, such as Color Flow Imaging (CFI) and Color Power Angio (CPA), suffer from the same speckle noise and shadowing artefact than B-mode imaging. However because of frame rate limitations, spatial compounding is not readily applied to flow imaging. For example, U.S. Pat. No. 6,390,980 (“the '980 patent”) teaches that conventional spatial compounding can be applied to Doppler signal information to derive Doppler power at receive angles close to zero, i.e., flow or motion orthogonal to the transmit beam provide no Doppler shift. The techniques disclosed in the '980 patent, however, reduce drastically the frame rate, so its real-time implementation is very limited. In particular, the '980 patent teaches that different look directions are acquired at different times, and the flow waveforms exhibit high acceleration (during systole). Applicants herein believe that the '980 patented techniques will not provide a desirable representation of the flow pattern throughout the cardiac cycle. More particularly, where conventional CFI and CPA are utilized, the different look directions create different velocity projections and therefore different velocity values. The different velocity values must be corrected before compounding.

Because flow imaging also suffers from shadowing and speckle noise, the present inventions provide new techniques perform real time spatial compounding in color flow imaging and CPA without compromising frame rate. The inventive techniques use different configurations of receive subapertures to achieve spatial compounding, wherein identical velocity projections of the Doppler signals are created for each of the different looks (angles), simultaneously, as distinguished, for example, from copending and commonly-owned U.S. Pat. No. 6,464,638. The contemporaneous available different looks, in accord with the receive subapertures configurations as taught hereby, provide a basis for real time CFI and CPA compounding imaging without frame rate limitation, realizing identical velocity projections of the Doppler signal for different looks.

Architecturally, the ultrasound imaging system may include a phased, linear, or curved linear array transducer in electrical communication with an ultrasound system controller configured to generate and forward a series of excitation signals to the transducer. The ultrasound imaging system may work in conjunction with the transducer to transmit ultrasound energy into a region of interest in a patient's body along a plurality of transmit lines. A transmit scan beam may be defined by a plurality of transmit scan lines. The ultrasound imaging system, may further comprise a receiver for receiving ultrasound echoes with the transducer from the region of interest in response to the ultrasound energy and for generating received signals representative of the received ultrasound echoes.

The system may also comprise a parallel beamformer for processing a plurality of received signals to form first and second sets of received ultrasonic beams, which originate at first and second spatially separated vantage points, respectively. In accordance with the present invention, a plurality of received ultrasonic scan beams may be steered and focused at multiple points along the transmit scan beam to simultaneously generate first and second beamformer signals representative of ultrasound echoes received along each of the transmit lines.

Other features and advantages of the invention will become apparent to one skilled in the art upon examination of the following drawings and detailed description. These additional features and advantages are intended to be included herein within the scope of the present invention.

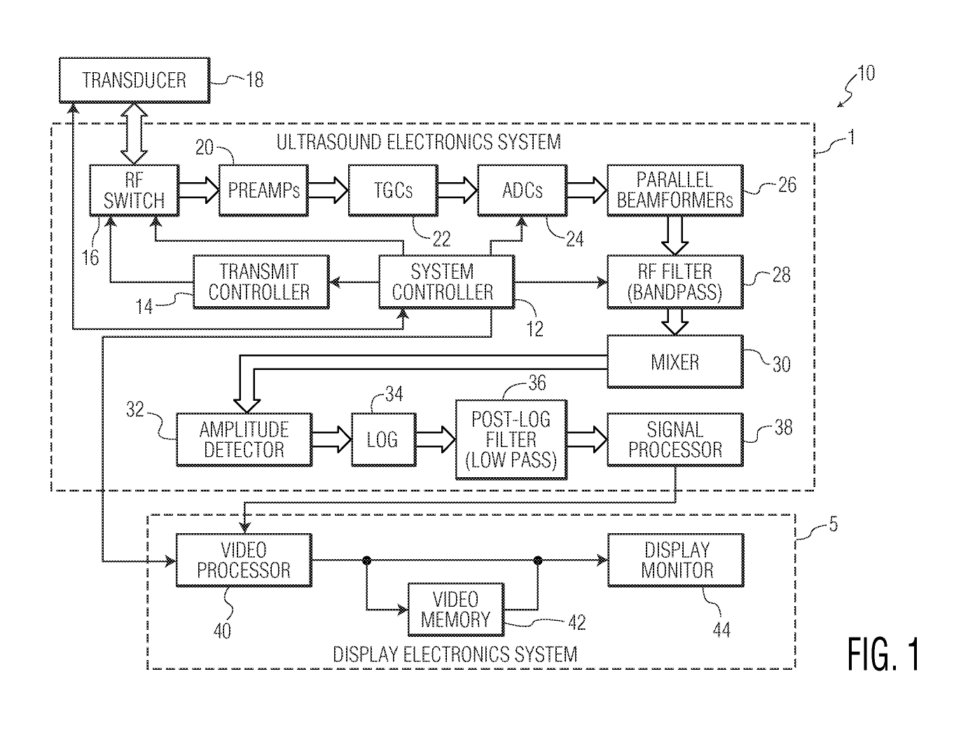

FIG. 1 is a block diagram of an ultrasound imaging system in accordance with the present invention that may practice the method of the present invention;

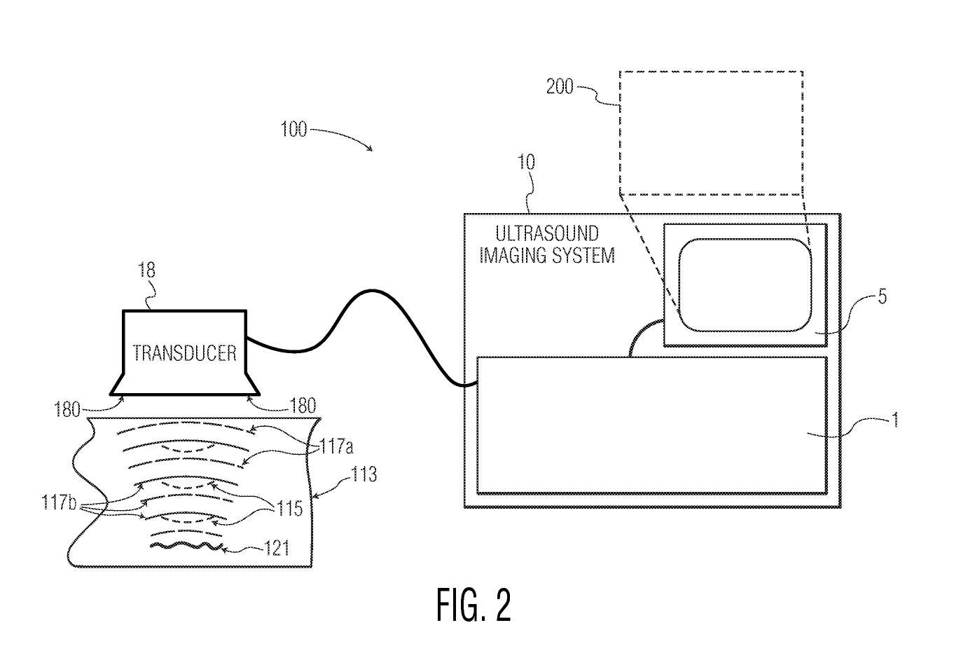

FIG. 2 is a diagram illustrating the use of the ultrasound imaging system of FIG. 1, in a medical diagnostic environment;

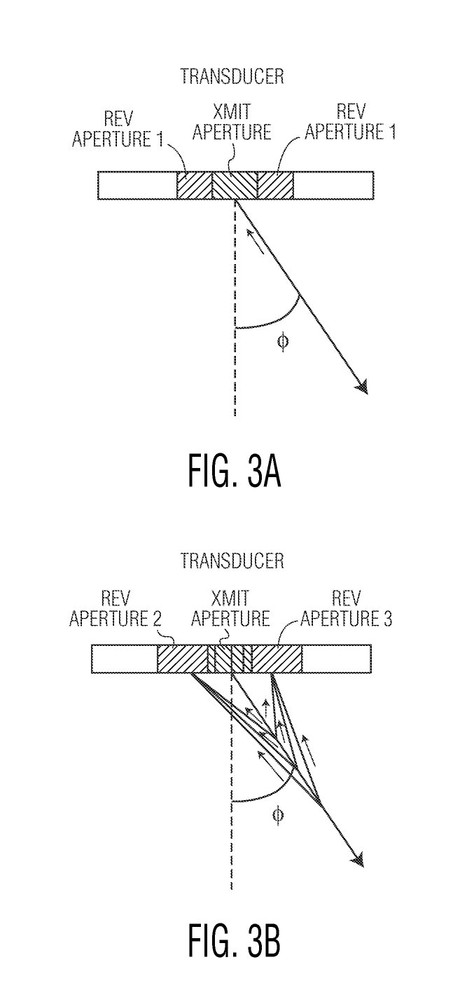

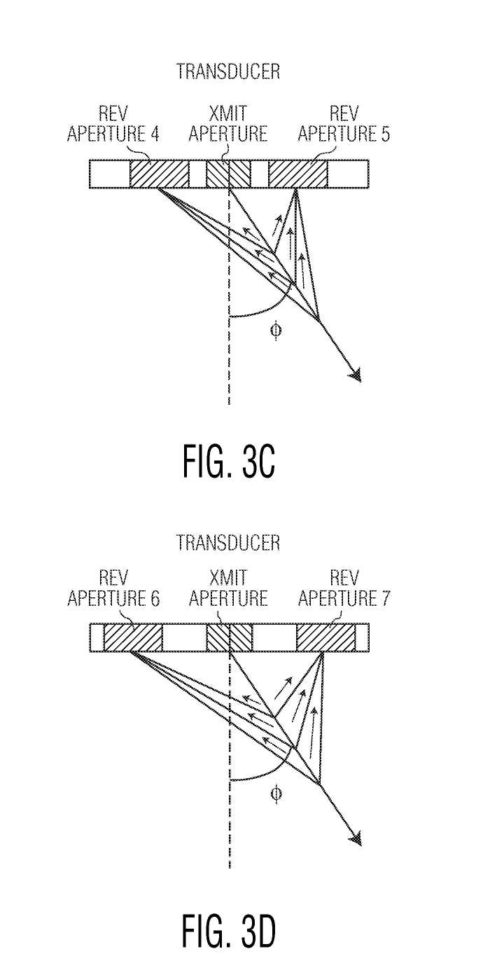

FIGS. 3A-3D are a set of related screenshots which depict color flow images of a flow phantom reconstructed from per channel data;

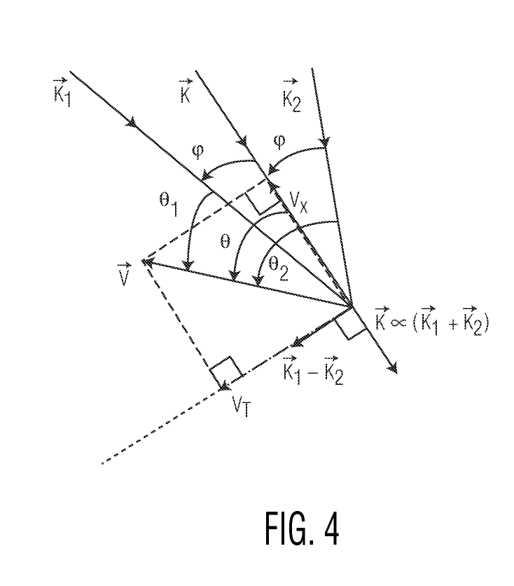

FIGS. 4A-4D are a set of related screenshots, which depict color flow images of a flow phantom reconstructed from per channel data;

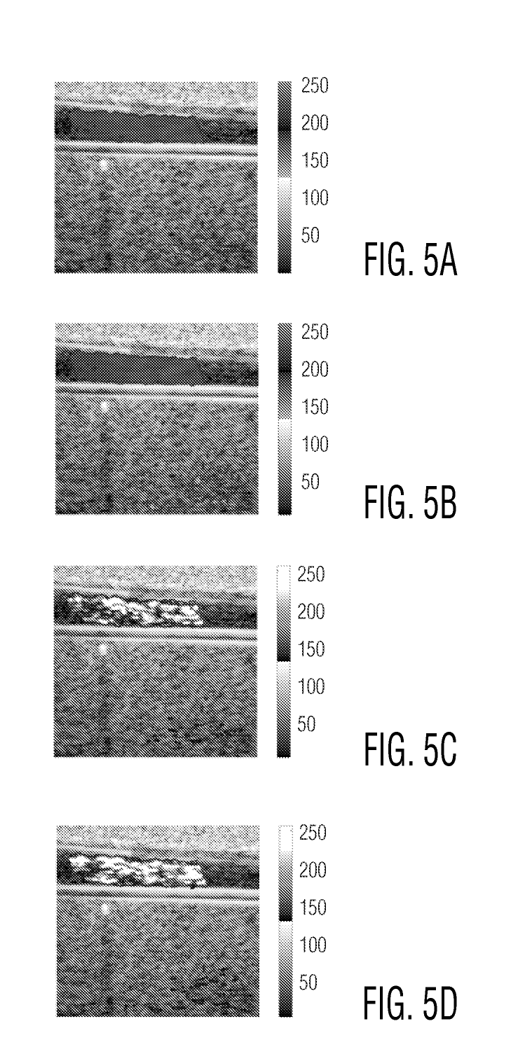

FIGS. 5A-5D are a set of related screenshots which, when viewed together, highlight the differences between conventional color flow imaging, and imaging realized with the inventive compounding methods taught hereby;

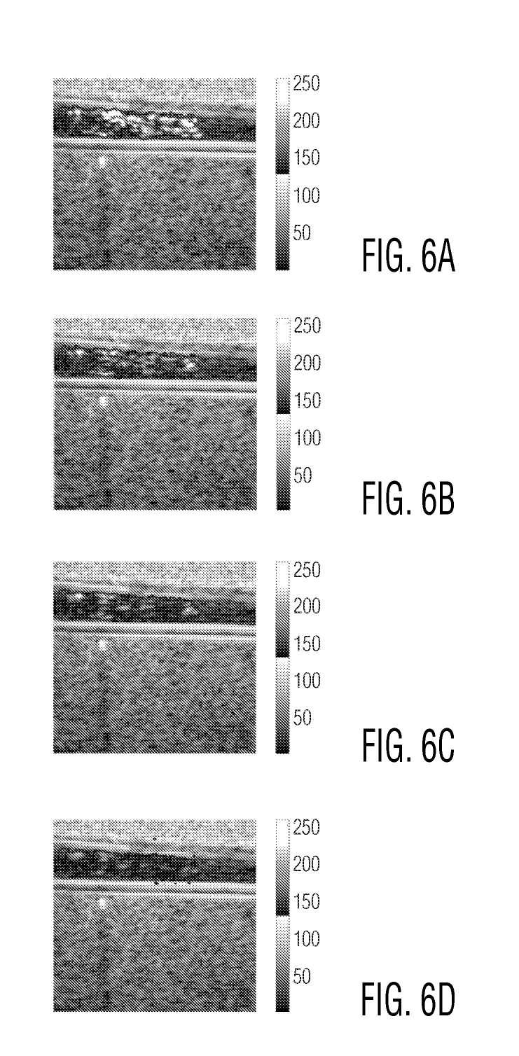

FIG. 6 is a diagram representative of math utilized by inventions herein.

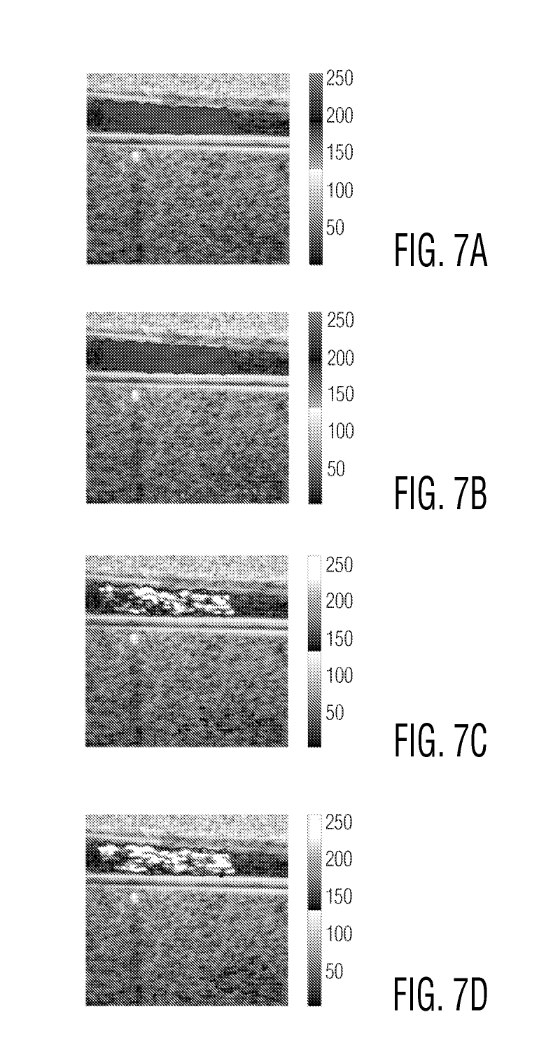

FIGS. 7A-7D depict a conventional Color flow image, a compounded Color flow image, a Conventional CPA image, and a compounded CPA image.

The improved ultrasound imaging system and method of the present invention will now be specifically described in detail in the context of an ultrasound imaging system that creates and displays brightness mode (B-Mode) images, or gray-scale images, which are well known in the art. However, it should be noted that the ultrasound imaging system and method of the present invention may be incorporated in other ultrasound imaging systems, including but not limited to, flow imaging systems, i.e., CFI and CPA, and other ultrasound imaging systems that are suited for the method, as will be apparent to those skilled in the art.

The present invention will be more fully understood from the detailed description given below and from the accompanying drawings of the preferred embodiment of the invention, which however, should not be taken to limit the invention to the specific embodiments enumerated, but are for explanation and for better understanding only. Furthermore, the drawings are not necessarily to scale, emphasis instead being placed upon clearly illustrating the principles of the invention. Finally, like reference numerals in the figures designate corresponding parts throughout the several drawings.

System Architecture and Operation

The architecture of an ultrasound imaging system capable of implementing the method of the present invention is illustrated by way of a functional block diagram in FIG. 1, and is generally denoted herein after by reference numeral 10. Note that many of the functional blocks illustrated in FIG. 1 define a logical function that can be implemented in hardware, software, or a combination thereof. For purposes of achieving high speed, it is preferred, at present, that most of the blocks be implemented in hardware, unless specifically noted hereafter.

Referring to FIG. 1, the ultrasound imaging system 10, may include an ultrasound electronics system 1, in communication with a transducer 18, and display electronics system 5. Ultrasound electronics system 1 may include a system controller 12 designed to control the operation and timing of the various elements and signal flow within the ultrasound imaging system 10, pursuant to suitable software. The ultrasound electronics system 1 may further comprise a transmit controller 14, a radio-frequency (RF) switch 16, a plurality of preamps 20, time-gain compensators (TGCs) 22, and analog to digital converters (ADCs) 24. In addition, the ultrasound electronics system 1 may comprise a parallel beamformer 26, a RF filter 28, a mixer 30, an amplitude detector 32, a log mechanism 34, a post-log filter 36, and a signal processor 38, video processor 40, a video memory device 42, and a display monitor 44.

The transducer 18 is configured to emit and receive ultrasound signals, or acoustic energy, respectively, to and from an object under test (e.g., the anatomy of a patient when the ultrasound imaging system 10 is used in the context of a medical application). The transducer 18 is preferably a phased array transducer having a plurality of elements both in the lateral and elevation directions, the laments typically made of a piezoelectric material, for example but not limited to, lead zirconate titanate (PZT). Each element is supplied with an electrical pulse or other suitable electrical waveform, causing the elements to collectively propagate an ultrasound pressure wave into the object under test. Moreover, in response thereto, one or more echoes are reflected by the object under test, and are received by the transducer 18, which transforms the echoes into electrical signals for further processing.

The array of elements associated with the transducer 18 enable a beam, emanating from the transducer array, to be steered (during transmit and receive modes) through the object by delaying the electrical pulses supplied to the separate elements. When the transmit mode is active, an analog waveform is communicated to each transducer element, thereby causing a pulse to be selectively propagated in a particular direction, like a beam, through the object. When the receive mode is active, an analog waveform is received at each transducer element at each beam position. Each analog waveform essentially represents a succession of echoes received by the transducer element over a period of time as echoes are received along the single beam through the object. Time delays are applied to the signals from each element in order to form a narrow receive beam in the desired direction. The entire set of analog waveforms formed by both transmit and receive mode manipulations represents an acoustic line, and the entire set of acoustic lines represents a single view, or image, of an object and is referred to as a frame.

As is known, a phased-array transducer may comprise a host of internal electronics responsive to one or more control signals that may originate within the system controller 12 or alternatively in the transmit controller 14. For example, the transducer electronics may be configured to select a first subset of transducer elements to apply an excitation signal in order to generate a plurality of ultrasonic pulses. In a related manner, the transducer electronics may be configured to select a second subset of transducer elements to receive ultrasonic echoes related to the transmitted ultrasonic pulses. Each of the aforementioned transducer element selections may be made by the transducer 18 in response to the one or more control signals originating in the transmit controller 14 or the system controller 12.

The transmit controller 14 may be electrically connected to the transducer 18 via a RF switch 16, and may be in further communication with the system controller 12. The system controller 12 may be configured to send one or more control signals in order to direct operation of the transmit controller 14, which in response generates a series of electrical pulses that may be periodically communicated to a portion of the array of elements of the transducer 18 via the RF switch 16, causing the transducer elements to emit ultrasound signals into the object under test of the nature described previously. The transmit controller 14 typically provides separation between the pulsed transmissions to enable the transducer 18 to receive echoes from the object during the period therebetween and forwards them onto a set of parallel analog preamplifiers 20, herein labeled, “PREAMPs.” The RF switch 16 may be configured to direct the various transmit and receive electrical signals to and from the transducer 18.

The plurality of preamplifiers 20 may receive a series of analog electrical echo waveforms from the transducer 18 that are generated by echoes reflected from the object under test. More specifically, each preamplifier 20 receives an analog electrical echo waveform from a corresponding set of transducer elements for each acoustic line. Moreover, the set of preamplifiers 20 receives a series of waveform sets, one set for each separate acoustic line, in succession over time and may process the waveforms in a pipeline processing manner. The set of preamplifiers 20 may be configured to amplify the echo waveforms to provide amplified echo waveforms in order to enable further signal processing, as described hereafter. Because the ultrasound signals received by the transducer 18 are of low power, the set of preamplifiers 20 should be of sufficient quality that excessive noise is not generated in the process.

Because the echo waveforms typically decay in amplitude as they are received from progressively deeper depths in the object under test, the plurality of analog preamplifiers 20 in the ultrasound electronics system 1 may be connected respectively to a parallel plurality of TGCs 22, which are known in the art and which are designed to progressively increase the gain during each acoustic line, thereby reducing the dynamic range requirements on subsequent processing stages. Moreover, the set of TGCs 22 may receive a series of waveform sets, one set for each separate acoustic line, in succession over time and may process the waveforms in a pipeline processing manner.

A plurality of parallel analog-to-digital converters (ADCs) 24 may be in communication respectively with the plurality of TGCs 21, as shown in FIG. 1. Each of the ADCs 22 may be configured to convert its respective analog echo waveform into a digital echo waveform comprising a number of discrete location points (hundreds to thousands; corresponding with depth and may be a function of ultrasound transmit frequency or time) with respective quantized instantaneous signal levels, as is well known in the art. In previous prior art ultrasound imaging systems, this conversion often occurred later in the signal processing steps, but now, many of the logical functions that are performed on the ultrasonic signals can be digital, and hence, the conversion is preferred at an early stage in the signal processing process. Similar to the TGCs 22, the plurality of ADCs 24 may receive a series of waveforms for separate acoustic lines in succession over time and process the data in a pipeline processing manner. As an example, the system may process signals at a clock rate of 40 MHz with a B-mode frame rate of 60 Hz.

A set of parallel beamformers 26 may be in communication with the plurality of ADCs 24 and may be designed to receive the multiple digital echo waveforms (corresponding with each set of transducer elements) from the ADCs 24 and combine them to form a single acoustic line. To accomplish this task, each parallel beamformer 26 may delay the separate echo waveforms by different amounts of time and then may add the delayed waveforms together, in order to create a composite digital RF acoustic line. The foregoing delay and sum beamforming process is well known in the art. Furthermore, the parallel beamformer 26 may receive a series of data collections for separate acoustic lines in succession over time and process the data in a pipeline processing manner.

An RF filter 28 may be coupled to the output of the parallel beamformers 26 and may be configured to receive and process a plurality of digital acoustic lines in succession. The RF filter 28 may be in the form of a bandpass filter configured to receive each digital acoustic line and to remove undesired out of band noise. As further illustrated in FIG. 1, a mixer 30 may be coupled at the output of the RF filter 28. The mixer 30 may be designed to process a plurality of digital acoustic lines in a pipeline manner. The mixer 30 may be configured to combine the filtered digital acoustic lines from the RF filter 28 with a local oscillator signal (not shown for simplicity) in order to ultimately produce a plurality of baseband digital acoustic lines. Preferably, the local oscillator signal is a complex signal, having an in-phase signal (real) and a quadrature phase signal (imaginary) that are ninety degrees out of phase. The result of the mixing operation may produce sum and difference frequency signals. The sum frequency signal may be filtered (removed), leaving the difference frequency signal, which is a complex signal at near zero frequency. A complex signal is desired in order to follow direction of movement of anatomical structures imaged in the object under test, and to allow accurate, wide bandwidth amplitude detection.

Up to this point in the ultrasound echo receive process, all operations can be considered substantially linear, so that the order of operations may be rearranged while maintaining substantially equivalent function. For example, in some systems it may be desirable to mix to a lower intermediate frequency (IF) or to baseband before beamforming or filtering. Such rearrangements of substantially linear processing functions are considered to be within the scope of this invention. An amplitude detector 32 may receive and process, in pipeline manner, the complex baseband digital acoustic lines from the mixer 30. For each complex baseband digital acoustic line, the amplitude detector 32 may analyze the envelope of the line to determine the signal intensity at each point along the acoustic line to produce an amplitude-detected digital acoustic line. Mathematically, this means that the amplitude detector 32 determines the magnitude of each phasor (distance to origin) corresponding with each point along the acoustic line.

A log mechanism 34 may receive the amplitude-detected digital acoustic lines in a pipeline processing manner, from the amplitude detector 32. The log mechanism 34 may be configured to compress the dynamic range of the data by computing the mathematical logarithm (log) of each acoustic line to produce a compressed digital acoustic line for further processing. Implementation of a log function enables a more realistic view, ultimately on a display, of the change in brightness corresponding to the ratio of echo intensities. A post-log filter 36, usually in the form of a low-pass filter, may be coupled to the output of the log mechanism 34 and may be configured to receive the compressed digital acoustic lines in a pipeline fashion. The post-log filter 36 may remove or suppress high frequencies associated with the compressed digital acoustic lines in order to enhance the quality of the ultimate display image. Generally, the post-log filter 36 softens the speckle in the displayed image. The low-pass post-log filter 36 can also be configured to perform anti-aliasing. The low-pass post-log filter 36 can be designed to essentially trade spatial resolution for gray-scale resolution.

A signal processor 38 may be coupled to the output of the low-pass post-log filter 36. The signal processor 38 may further comprise a suitable species of random access memory (RAM) and may be configured to receive the filtered digital acoustic lines from the low-pass post-log filter 36. The acoustic lines can be defined within a two-dimensional coordinate space. The signal processor 38 may be configured to mathematically manipulate image information within the received and filtered digital acoustic lines. In an alternative embodiment, the signal processor 38 may be configured to accumulate acoustic lines of data over time for signal manipulation. In this regard, the signal processor 38 may further comprise a scan converter to convert the data as stored in the RAM in order to produce pixels for display. The scan converter may process the data in the RAM once an entire data frame (i.e., a set of all acoustic lines in a single view, or image/picture to be displayed) has been accumulated by the RAM. For example, if the received data is stored in RAM using polar coordinates to define the relative location of the echo information, the scan converter may convert the polar coordinate data into rectangular (orthogonal) data capable of raster scan via a raster scan capable processor.

Having completed the receiving, echo recovery, and signal processing functions, to form a plurality of image frames associated with the plurality of ultrasound image planes, the ultrasound electronics system 1, may spatially compound the plurality of image frames by mathematically combining (e.g., averaging) the plurality of image frames to form a single image frame with reduced speckle. Various conventional methods are known to the skilled artisan.

Having spatially compounded the plurality of image frames, the ultrasound electronics system 1 may forward the echo image data information associated with the single spatially compounded image frame to a display electronics system 5, as illustrated in FIG. 1. The display electronics system 5 may receive the echo image data from the ultrasound electronics system 1, where the echo image data may be forwarded to a video processor 40. The video processor 40 may be designed to receive the echo image data information and may be configured to raster scan the image information.

The video processor 40 outputs picture elements (e.g., pixels) for storage in a video memory device 42 and/or for display via a display monitor 44. The video memory device 42 may take the form of a digital video disk (DVD) player/recorder, a compact disc (CD) player/recorder, a video cassette recorder (VCR) or other various video information storage devices. As is known in the art, the video memory device 42 permits viewing and or post data collection image processing by a user/operator in other than real-time. A conventional display device in the form of a display monitor 44 may be in communication with both the video processor 40 and the video memory 42 as illustrated in FIG. 1. The display monitor 44 may be configured to periodically receive the pixel data from either the video memory 42 and or the video processor 40 and drive a suitable screen or other imaging device (e.g., a printer/plotter) for viewing of the ultrasound image by a user/operator.

Fundamental Image Formation

Having described the architecture and operation of the ultrasound imaging system 10 of FIG. 1, attention is now directed to FIG. 2, which illustrates the general diagnostic environment 100, where the ultrasound imaging system 10 of FIG. 1 may use the method of the present invention to improve a two-dimensional ultrasound image. The diagnostic environment 100 comprises a patient under test 113, and a transducer 18. The transducer 18 may be placed into position over a portion of the anatomy of a patient under test 113 by a user/operator (not shown), and a plurality of transmit pulses 115 are transmitted from the transducer. When the transmit pulses (ultrasound energy) 115 encounter a tissue layer of the patient under test 113 that is receptive to ultrasound insonification, the multiple transmit pulses 115 penetrate the tissue layer 113.

As long as the magnitude of the multiple ultrasound pulses exceeds the attenuation affects of the tissue layer 113, the multiple ultrasound pulses 115 will reach an internal target 121. Those skilled in the art will appreciate that tissue boundaries or intersections between tissues with different ultrasonic impedances will develop ultrasonic responses at the fundamental transmit frequency of the plurality of ultrasound pulses 115. Tissue insonified with ultrasonic pulses will develop fundamental ultrasonic responses that may be distinguished in time from the transmit pulses in order to convey information from the various tissue boundaries within a patient.

Those ultrasonic reflections of a magnitude that exceed that of the attenuation affects from traversing tissue layer 113 may be monitored and converted into an electrical signal by the combination of the RF switch 16 and the transducer 18, as previously described with regard to FIG. 1. The ultrasound electronics system 1, and the display electronics system 5, may work together to produce an ultrasound display image 200, derived from the plurality of ultrasonic echoes 117.

The new approach of the present inventions includes use of a single transducer array, transmitting ultrasound from a single aperture and receiving backscattered echoes from several sub-arrays defined by contiguous sets of elements, on either side of the transmit aperture. That is, the present invention includes insonifying a target image with ultrasonic energy, and receiving or capturing the target image from a number of different vantage points, distinguished by angle, simultaneously, and mathematically combining the different images to reduce the speckle. By mathematically combining (e.g., averaging) a plurality of images formed from information gathered from a number of vantage points, the speckle patterns lack correlation, while the target echoes remain correlated and virtually unchanged.

FIGS. 3A-3D illustrate different configurations of transmit/receive aperture configurations, which may be implemented by the present inventions. That is, FIGS. 3A-3D depicts color Flow images of a flow phantom reconstructed from per channel RF data. FIG. 3A depicts using a conventional receive configuration, where FIG. 3B depicts using a receive configuration where φ₁=2.5°. FIG. 3C depicts using receive configuration where φ₂=5°, and

FIG. 3D depicts using receive configuration where φ₃=7.5°.

FIG. 4 illustrates the basic math required, where \vec{K} is a unit vector in the direction of the transmitted ultrasound beam, and \vec{K₁} and \vec{K₂} are unit vectors parallel to the two receiving directions of the two sub arrays. If the position of the left and right sub-aperture centers are such that the vectors \vec{K₁} and \vec{K₂} subtend the same angle φ with respect to the transmit wave vector \vec{K}, the vector sum [symbol] is parallel to the transmit beam-steering direction and therefore to \vec{K}. If scatterers move with a velocity \vec{V} past a sample volume in the insonified field of view, the mean Doppler frequency shift received by the sum of the two receive subapertures is proportional to the velocity projection Vₓ on vector \vec{K}:

$${V}_{x} = \vert \vert \vec{V} cos ( θ ) = \frac{( \vec{{K}_{1} + {K}_{2} ) \cdot V + 2 K \cdot V}}{2 c ( o ϕ ) s + 2} \qquad ( 1 )$$

where θ is the angle between the transmit beam and the velocity vector \vec{V} and φ is the angle between the receive and transmit beams

As a result of the lack of correlation in the speckle patterns between the various vantage points, the variance in the speckle patterns can be reduced without degrading the target image. The calculations to mathematically combine images formed from different vantage points for reducing speckle are well known. Typically, the way to generate multiple images from different directions with a “fixed” transducer is to excite different cells or groups of cells of a linear or curved linear array of piezoelectric transducer elements, which are used to generate and receive the ultrasound energy. The vantage point for an ultrasound beam is typically controlled by the physical position of an active aperture used for forming the ultrasound beam. Thus the groups in a fixed transducer must be separated along the array in order to achieve the required spatially separated vantage points.

By way of example, one can separate a linear array of N transducer elements into M sections, each section having N/M contiguous transducer elements and defined by a unique location or vantage point along the array. Each section may be electrically excited one at a time in succession with the resulting ultrasound beam from each of the transducer sections steered so that all M beams are focused at substantially the same region, but from different directions having their origin at the face of the transducer array. Speckle can then be reduced by combining the M ultrasound beams (controlled by both transmit and receive processing) from the related M different vantage points.

FIGS. 5A-5D depict CPA images of a flow phantom that were reconstructed from per channel RF data. That is, FIG. 5A depicts an image reconstructed from data received through a conventional receive configuration, where FIG. 5B depicts a reconstructed image from data received using the inventive receive structures, where φ₁=2.5°, FIG. 5C depicts receive configuration where φ₂=5°, and FIG. 5D depicts using receive configuration where φ₃=7.5°.

Further screen shots of images reconstructed from CFI and CPA flow data in accord with the inventions herein are shown in FIGS. 6A-6D. In particular, FIG. 6B depicts the compounded CFI image having fewer holes, and shows more regular delineation of the flow within the vessel lumen than conventional flow image (FIG. 6A). The compounded CPA of FIG. 6D shows reduced speckle pattern and a better “filling” of the vessel lumen than conventional CPA (FIG. 6C) without too much degradation of the lateral resolution. As can be readily understood from a review of FIGS. 6A-6B, the inventive techniques offer a compromise between spectral broadening (due to the size of the apertures) and lateral resolution. This technique will also improved to sensitivity of color flow imaging.

This inventive approach may be implemented in the Boris platform by trading off multiline factor with compounding angles. In the case of a 4× multiline factor no compounding could be applied. In the case of 2× multiline, 2 compounded angles could be achieved (conventional configuration+one of the b, c or d configuration). In the case of no multiline, 4 compounded 4 angles could be used. For that matter, the inventions map better to the Boris plus architecture because the QSC has 16 parallel receive paths and therefore for a one-D array, it will be possible to achieve 4× multiline with 4 compounding angles concurrently. Those skilled in the art will understand that modifying Boris to implement the present inventions requires preparation of “new” acquisition tables. One skilled in the art, and understanding the proprietary Boris platform, will also understand that new or revised acquisition tables would be required to be defined in order to support the new receive aperture configurations as described herein, and that the FEC will be required to load the inventive aperture arrangement. The skilled artisan will also understand that the platform is not a limitation of the inventions, and that any platform which can support the receive aperture arrangement, and processing of data therefrom, will be able to implement the improved compounding in flow imaging as taught and claimed hereby.

Back to the Boris platform example, the DSC architecture need not be changed because the different “look” angles may be processed as a derivative multiline. Of course it should be readily understood that different normalization functions would be required to be applied to the different receive configurations. For that matter, Philips proprietary Boris SIP would require modification to compound the different angles before performing regular color flow/CPA processing.

FIGS. 7A-7D depict a conventional Color flow image, a compounded Color flow image, a Conventional CPA image, and a compounded CPA image, respectively, to highlight the difference in image quality in before and after screenshots. That is, the four figures provides an understanding of the results and benefits of the compounding to remove speckle during flow processing as implemented in accordance with the inventions. For that matter, the inventions disclosed hereby are particularly suited for shallow vascular applications, for example, in the presence of stenosis where the plaque can create shadowing, in small vessel imaging such as in the thyroid or in the breast.

It is significant to note that software required to perform the functional activities as illustrated, and or the mathematical combinations and data manipulations necessary to spatial compound ultrasound images in two-dimensions may comprise an ordered listing of executable instructions for implementing logical functions. As such, the software can be embodied in any computer-readable medium for use by or in connection with an instruction execution system, apparatus, or device, such as a computer-based system, processor-containing system, or other system that can fetch the instructions from the instruction execution system, apparatus, or device and execute the instructions. In the context of this document, a “computer-readable medium” can be any means that can contain, store, communicate, propagate, or transport the program for use by or in connection with the instruction execution system, apparatus, or device.

The computer readable medium can be, for example but not limited to, an electronic, magnetic, optical, electromagnetic, infrared, or semiconductor system, apparatus, device, or propagation medium. More specific examples (a nonexhaustive list) of the computer-readable medium would include the following: an electrical connection (electronic) having one or more wires, a portable computer diskette (magnetic), a random access memory (RAM) (magnetic), a read-only memory (ROM) (magnetic), an erasable programmable read-only memory (EPROM or Flash memory) (magnetic), an optical fiber (optical), and a portable compact disc read-only memory (CDROM) (optical). Note that the computer-readable medium could even be paper or another suitable medium upon which the program is printed, as the program can be electronically captured, via for instance optical scanning of the paper or other medium, then compiled, interpreted or otherwise processed in a suitable manner if necessary, and then stored in a computer memory.

It should be emphasized that the above-described embodiments of the present invention, particularly, any “preferred” embodiment(s), are merely possible examples of implementations that are merely set forth for a clear understanding of the principles of the invention. Furthermore, many variations and modifications may be made to the above-described embodiments of the invention without departing substantially from the spirit and principles of the invention. All such modifications and variations are intended to be taught by the present disclosure, included within the scope of the present invention, and protected by the following claims.

1. An ultrasound imaging system, comprising:
a transmitter configured to generate a plurality of time interleaved transmit signals;
a transducer in communication with the transmitter, and configured to translate the plurality of time-interleaved signals, and transmit said signals through a single aperture;
a receiver in communication with the transducer, and configured to receive the plurality of receive signals at two or more sub apertures, said sub-apertures defined by contiguous sets of transducer elements, where said elements are disposed on either side of said single aperture, and acquires a plurality of response scan beams, at varying angles, simultaneously, using a beamforming technique;
a signal processor in communication with the receiver configured to mathematically combine image information derived from the plurality of response scan beams into a display signal; and
a monitor in communication with the signal processor configured to convert the display signal into an image.

2. The system of claim 1, wherein the transducer comprises at least a first receive aperture and a second receive aperture, such that a look direction is formed from each of the first and second apertures.

3. The system of claim 1, wherein the transducer is a phased-array transducer.

4. The system of claim 1, wherein the transducer is a linear-array transducer.

5. The system of claim 4, wherein the transducer is a curved linear-array transducer.

6. A method for reducing speckle in an ultrasound image, comprising the following steps:
generating a transmit scan beam from a single aperture defined on a face of a transducer element array, such that the transmit scan beam originates from the single aperture;
generating a first set of ultrasound response scan beams, originating from a first receive aperture, defined as a first set of transducer elements symmetrically across the center of the transmit aperture;
generating at least a second set of ultrasound response scan beams, originating from at least a second receive aperture contiguous with the first receive aperture, wherein the at least second receive aperture is defined by at least a second set of transducer elements, the at least second set of transducer elements disposed symmetrically across the center of the transmit aperture, wherein the response scan beams are received simultaneously by the first and the at least second receive apertures; and
compounding the image information.

7. The method of claim 6, wherein the step of recovering is performed with a beamforming technique.

8. The method of claim 7, wherein the step of compounding is performed in conjunction with frequency compounding.

9. An ultrasound imaging system, comprising:
means for generating and transmitting a transmit scan beam from a single transmit aperture of a transducer array matrix;
means for generating a plurality of ultrasound response scan beams, each response scan beam originating from at least two receive sub-apertures which are contiguous with, and centered upon the single transmit aperture, such that response beams correlate to different look directions;
means for recovering image information derived from the plurality of look direction, simultaneously;
means for spatially compounding the recovered image information derived from the plurality of look direction, simultaneously, to realize spatially compounded image information; and
means for converting the spatially compounded image information such that an operator may view it.

10. The system of claim 9, wherein the means for recovering image information from the plurality of ultrasound response scan beams comprises a parallel beamforming technique.

11. The system of claim 9, wherein the means for generating a plurality of ultrasound response scan beams is accomplished with a one-dimensional mechanically scanned transducer array.

12. The system of claim 9, wherein the means for generating a plurality of ultrasound response scan beams is accomplished with an electronically manipulated two-dimensional array.

13. The system of claim 9, wherein the means for spatially compounding is further configured to perform elevation compounding in conjunction with at least one other method for compounding an image selected from the group consisting of lateral compounding and frequency compounding.

14. A computer readable medium comprising a set of computer-readable instructions, which set of instructions, when operated upon by a general purpose computer implements a method as set forth in claim 1.
